SEQUENCE LISTING (14 sequences)

SEQ ID NO: 1 - protein, 223 aa, Grifola frondosa
MGSSHHHHHHSSGLVPRGSGTMLYGVEIDEQYLRVMEEYKDKEVITQADMAKVALQRKNVYQDQAEKRQAELKAEYGVGVCVLVRVYNATGGPITAKIEESFRGHFGAHTREKRIGNGQWTVFIHTKSAGAAVGSAGCIVYGTTDNLDIFSGWQNPWNRSWDSQVLVEVRQSGHWWKNGSKDYMLHLLDTHNGQNSDSSYGDVKAHGSTGNETTAYVEYVYSR

SEQ ID NO: 2 - DNA, 672 bp, Grifola frondosa
atgggcagcagccatcatcatcatcatcacagcagcggcctggtgccgcgcggcagcggtaccatgctttacggcgtcgagatagacgagcagtacctgcgcgtgatggaggagtacaaggacaaggaagtaatcacgcaggccgacatggccaaggtcgcgctacaacgcaagaacgtgtaccaggaccaggccgagaagcgccaggctgagcttaaggccgaatatggagtcggtgtctgtgtcctcgtgcgtgtgtacaatgctactggcggcccgatcacagcgaagatcgaggagtccttccgaggccactttggcgcgcacactcgagaaaagcgcatcgggaatgggcaatggacggtgttcatccacaccaagtcggcgggcgccgcagtcgggtccgcggggtgcatcgtgtatggcaccacggacaacctcgacatcttctcaggctggcagaacccctggaaccgctcgtgggattctcaggtgctcgttgaggtgcgccagagcggtcattggtggaagaacggtagcaaggactatatgctccacctactcgacacgcacaacggccagaatagcgactcgagctacggcgatgtcaaggctcacgggtcgactggaaatgagactaccgcttacgtggagtatgtgtactcccgttga

SEQ ID NO: 3 - DNA, 22 bp, Artificial Sequence
  Description of Artificial Sequence  Synthetic nucleotide (5′-RACE-GF)
catgcgctagaacaagcaatac

SEQ ID NO: 4 - DNA, 22 bp, Artificial Sequence
  Description of Artificial Sequence  Synthetic nucleotide (3′-RACE-GF)
acaacctcgacatcttctcagg

SEQ ID NO: 5 - DNA, 20 bp, Artificial Sequence
  Description of Artificial Sequence  Synthetic nucleotide (Nni-F)
caccatgctttacggcgtcg

SEQ ID NO: 6 - DNA, 24 bp, Artificial Sequence
  Description of Artificial Sequence  Synthetic nucleotide (Nni-R)
tcaacgggagtacacatactccac

SEQ ID NO: 7 - protein, 472 aa, Artificial Sequence
  Description of Artificial Sequence  Synthetic polypeptide (GFP-GF-Nni)
MGSSHHHHHHSSGLVPRGSHMARVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLGGGGGEFMLYGVEIDEQYLRVMEEYKDKEVITQADMAKVALQRKNVYQDQAEKRQAELKAEYGVGVCVLVRVYNATGGPITAKIEESFRGHFGAHTREKRIGNGQWTVFIHTKSAGAAVGSAGCIVYGTTDNLDIFSGWQNPWNRSWDSQVLVEVRQSGHWWKNGSKDYMLHLLDTHNGQNSDSSYGDVKAHGSTGNETTAYVEYVYSR

SEQ ID NO: 8 - DNA, 1419 bp, Artificial Sequence
  Description of Artificial Sequence  Synthetic nucleotide (GFP-GF-Nni)
atgggcagcagccatcatcatcatcatcacagcagcggcctggtgccgcgcggcagccatatggctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttggaggaggaggaggagaattcatgctttacggcgtcgagatagacgagcagtacctgcgcgtgatggaggagtacaaggacaaggaagtaatcacgcaggccgacatggccaaggtcgcgctacaacgcaagaacgtgtaccaggaccaggccgagaagcgccaggctgagcttaaggccgaatatggagtcggtgtctgtgtcctcgtgcgtgtgtacaatgctactggcggcccgatcacagcgaagatcgaggagtccttccgaggccactttggcgcgcacactcgagaaaagcgcatcgggaatgggcaatggacggtgttcatccacaccaagtcggcgggcgccgcagtcgggtccgcggggtgcatcgtgtatggcaccacggacaacctcgacatcttctcaggctggcagaacccctggaaccgctcgtgggattctcaggtgctcgttgaggtgcgccagagcggtcattggtggaagaacggtagcaaggactatatgctccacctactcgacacgcacaacggccagaatagcgactcgagctacggcgatgtcaaggctcacgggtcgactggaaatgagactaccgcttacgtggagtatgtgtactcccgttga

SEQ ID NO: 9 - DNA, 30 bp, Artificial Sequence
  Description of Artificial Sequence  Synthetic nucleotide (5′-RACE-HM)
gttataatgcatgaacgcactccattggcc

SEQ ID NO: 10 - DNA, 30 bp, Artificial Sequence
  Description of Artificial Sequence  Synthetic nucleotide (3′-RACE-HM)
atgcgcgagagggataaatacgaaggggag

SEQ ID NO: 11 - DNA, 30 bp, Artificial Sequence
  Description of Artificial Sequence  Synthetic nucleotide (HM-F)
caccatgaaatacggactcatcatcgacaa

SEQ ID NO: 12 - DNA, 30 bp, Artificial Sequence
  Description of Artificial Sequence  Synthetic nucleotide (HM-R)
ctatgccttctcaataatagtgacacaggc

SEQ ID NO: 13 - protein, 209 aa, Hypsizygus marmoreus
MKYGLIIDNDYLRLLPEYKDKQDWQITVYDRSKVAMRERDKYEGEAITRTHLLKVNSGIGSVGASVECRVYNASGGRLRVHLTKSWSGDFYQDQPDHYLENGQWSAFMHYNDGWSGISSATGAVVYRTGSDTDIFIGWFTKRDVPMSPACYVESQGKDPWWNVGSEGFMEYLIKDKGDLRSRDRQRGYKVTTIIGQSCTSACVTIIEKA

SEQ ID NO: 14 - DNA, 827 bp, Hypsizygus marmoreus
taatacgactcactatagggcaagcagtggtatcaacgcagagtacatgggaacctcctcagaaagggctcacgcataggtcctcaaatccccaccttcactatgaaatacggactcatcatcgacaacgactatctcaggctgcttcctgagtacaaggacaaacaggattggcaaatcactgtgtatgatcgaagcaaggttgcaatgcgcgagagggataaatacgaaggggaggccataactcgcactcacctcctcaaagtcaactcgggtattggatcagtcggcgcatcagtggagtgccgggtctataacgcatcaggcggcagacttcgagtgcacctcacgaaatcgtggagtggtgatttctaccaggaccagccagaccactacctcgagaacggccaatggagtgcgttcatgcattataacgatggctggtcaggcatcagctctgccacgggtgctgttgtataccgcactgggtcagacacggacatcttcatcgggtggttcacaaagagagatgttccgatgagtcccgcgtgttacgtggagtcgcagggaaaggatccctggtggaatgtgggttcggaggggttcatggagtacttgatcaaagacaagggagacctgcgttctcgagatagacagcggggatataaggtaacgacgatcattgggcagtcgtgcactagcgcctgtgtcactattattgagaaggcatagatgaatagtccatttcgcgccacatcagaagcatccatttaatacacatcatgcaacagtgaaaaaaaaaaaaaaaaaaaaaaaaaaaaaaaaaa

(19) United States
(12) Patent Application Publication
(10) Pub. No.: US 2013/0115625 A1
(43) Pub. Date: May 9, 2013

(54) NOVEL PROTEIN AND USE THEREOF

(71) Applicants: Toshihide Kobayashi, Wako-shi (JP); Tetsuyuki Kobayashi, Bunkyo-ku (JP); Kozo Nishibori, Minamiuonuma-shi (JP); Atsushi Kurahashi, Minamiuonuma-shi (JP); Fumihiro Fujimori, Funabashi-shi (JP)

(72) Inventors: Toshihide Kobayashi, Wako-shi (JP); Tetsuyuki Kobayashi, Bunkyo-ku (JP); Kozo Nishibori, Minamiuonuma-shi (JP); Atsushi Kurahashi, Minamiuonuma-shi (JP); Fumihiro Fujimori, Funabashi-shi (JP)

(73) Assignees: RIKEN, Saitama (JP); YUKIGUNI MAITAKE CO., LTD., Niigata (JP); OCHANOMIZU UNIVERSITY, Tokyo (JP)

(21) Appl. No.: 13/697,402

(22) PCT Filed: May 13, 2011
(86) PCT No.: PCT/JP2011/061104

§ 371 Date: Jan. 25, 2013

(30) Foreign Application Priority Data
May 14, 2010 (JP) 2010112681

(51) Int. Cl. C07K 14/375; C07K 16/14

(52) U.S. Cl. CPC C07K 14/375; C07K 16/14
USPC 435/7.21; 435/7.92; 435/252.33; 435/254.11; 435/320.1; 436/501; 530/350; 530/387.9; 536/23.1

(57) ABSTRACT

A protein being the following (A), (B), or (C): (A) a protein represented by the amino acid sequence of SEQ ID NO: 1; (B) a protein represented by an amino acid sequence in which one or a plurality of amino acid is substituted, deleted, inserted or added in the amino acid sequence of SEQ ID NO: 1, the protein having binding activity specific for a mixture of a sphingolipid and cholesterol; or (C) a protein represented by an amino acid sequence being at least 70% identical to the amino acid sequence of SEQ ID NO: 1.

### TECHNICAL FIELD

The present invention relates to a novel protein having binding activity specific for a mixture of a sphingolipid and cholesterol, and relates to a use thereof.

### BACKGROUND ART

A lipid raft is a domain on a cell membrane. The lipid raft has a diameter of several 10 nm to 100 nm, and is mainly made up of a sphingolipid and cholesterol. It is considered that the lipid raft is involved in various physiological reactions that occur with respect to a cell membrane, such as important reactions for example signal transduction via the membrane, bacterial or virus infections, and intracellular traffic.

Patent Literature 1, the entire contents of which are hereby incorporated by reference, discloses a protein including a specific amino acid sequence of lysenins (a toxoprotein secreted from Eisenia foetida) as a protein which specifically recognizes sphingomyelin (one kind of the sphingolipid). Moreover, Patent Literature 2, the entire contents of which are hereby incorporated by reference, discloses a protein in which an N terminal and/or C terminal of earthworm toxin lysenin 1 or earthworm toxin lysenin 3 is deleted.

Moreover, Patent Literature 3, the entire contents of which are hereby incorporated by reference, discloses a method of detecting sphingomyelin, by (i) immobilizing lipids contained in a test sample to a solid phase and (ii) detecting lysenin attached to the solid phase.

Patent Literature 4, the entire contents of which are hereby incorporated by reference, discloses polyethylene glycol cholesteryl ether as a substance specifically recognizing cholesterol. Patent Literature 5, the entire contents of which are hereby incorporated by reference, discloses polyethylene glycol 2-aminoethyl cholesteryl ether as a substance specifically recognizing cholesterol. Moreover, these literatures further disclose cholesterol detection reagents which contain these substances, respectively.

### CITATION LIST

### Patent Literatures

Patent Literature 1

Japanese Patent Application Publication, Tokukai, No. 2002-355035 A (Publication Date: Dec. 10, 2002)

Patent Literature 2

Japanese Patent Application Publication, Tokukai, No. 2003-061680 A (Publication Date: Mar. 4, 2003)

Patent Literature 3

Japanese Patent Application Publication, Tokukai, No. 2006-214731 A (Publication Date: Aug. 17, 2006

Patent Literature 4

Japanese Patent Application Publication, Tokukai, No. 2003-344419 A (Publication Date: Dec. 3, 2003)

Patent Literature 5

Japanese Patent Application Publication, Tokukai, No. 2004-354284 A (Publication Date: Dec. 16, 2004

### SUMMARY OF INVENTION

### Technical Problem

However, the conventional techniques described above detect sphingomyelin alone or cholesterol alone, and not those in the form of lipid raft. Hence, the conventional techniques have a problem in that it is not possible to specifically detect a lipid raft by the conventional techniques.

It is necessary to perform a study on a distribution, kinetics, a function, and the like of the lipid raft for elucidating a reaction mechanism with which a lipid raft is involved. In order to do so, a technique for specifically detecting a lipid raft has been demanded.

The present invention is accomplished in view of the foregoing problem, and an object thereof is to provide a novel protein which allows for specifically detecting a lipid raft.

### Solution to Problem

As a result of diligent study to attain the foregoing object, the inventors of the present invention found a novel protein derived from Hen-of-the-woods, which novel protein specifically binds to a mixture of a sphingolipid and cholesterol, and accomplished the present invention based on the finding.

Namely, a protein according to the present invention is a protein being the following (A), (B), or (C).

(A) A protein represented by the amino acid sequence of SEQ ID NO: 1;

(B) A protein represented by an amino acid sequence in which one or a plurality of amino acid is substituted, deleted, inserted or added in the amino acid sequence of SEQ ID NO: 1, the protein having binding activity specific for a mixture of a sphingolipid and cholesterol; or

(C) A protein represented by an amino acid sequence being at least 70% identical to the amino acid sequence of SEQ ID NO: 1, the protein having binding activity specific for a mixture of a sphingolipid and cholesterol.

Moreover, a polynucleotide according to the present invention codes for the foregoing protein.

Moreover, it is preferable that a polynucleotide according to the present invention is a polynucleotide being the following (A), (B), (C), or (D).

(A) A polynucleotide represented by the base sequence of SEQ ID NO: 2;

(B) A polynucleotide represented by a base sequence in which one or a plurality of base is substituted, deleted, inserted or added in the base sequence of SEQ ID NO: 2, the polynucleotide coding for a protein having binding activity specific for a mixture of a sphingolipid and cholesterol;

(C) A polynucleotide that hybridizes, under a stringent condition, with a polynucleotide consisting of a base sequence complementary to the base sequence of SEQ ID NO: 2, the polynucleotide coding for a protein having binding activity specific for a mixture of a sphingolipid and cholesterol; or

(D) A polynucleotide represented by a base sequence being at least 70% identical to the base sequence of SEQ ID NO: 2, the polynucleotide coding for a protein having binding activity specific for a mixture of a sphingolipid and cholesterol.

Moreover, a vector according to the present invention includes the foregoing polynucleotide.

Moreover, a transformant according to the present invention has the foregoing polynucleotide introduced in the transformant.

Moreover, the transformant according to the present invention has the vector introduced in the transformant.

Moreover, an antibody according to the present invention binds to the foregoing protein.

Moreover, a method according to the present invention of detecting a mixture of a sphingolipid and cholesterol uses the foregoing protein.

Moreover, a kit according to the present invention of detecting a lipid raft includes at least one selected from the group consisting of: the protein, the polynucleotide, the vector, the transformant, and the antibody.

Moreover, a protein according to the present invention is a homologue of a protein represented by the amino acid sequence of SEQ ID NO: 1.

A virus infection inhibitor according to the present invention includes the protein.

### Advantageous Effects of Invention

The protein according to the present invention has binding activity specific for a mixture of sphingolipid and cholesterol. Hence, it is possible to specifically detect a lipid raft.

Other objects, features, and outstanding points of the present invention are fully understandable by the following descriptions. Moreover, advantages of the present invention are made clear by the following descriptions with reference to the accompanying drawings.

### BRIEF DESCRIPTION OF DRAWINGS

FIG. 1 is a view showing a result of detecting GF-Nni by electrophoresis, by mixing (i) a protein GF-Nni according to an example of the present invention and (ii) various liposomes and separating this mixture into supernatants (S) and precipitates (P).

FIG. 2 is a graph showing binding activities of GF-Nni for mixtures of lipids and cholesterol.

FIG. 3 is a graph showing binding activities of GF-Nni for mixtures of sphingomyelin (S) and cholesterol (C).

FIG. 4 is a graph showing binding activities of GF-Nni for mixtures of various sphingomyelins and cholesterol.

FIG. 5 is a graph showing binding activities of GF-Nni for mixtures of sphingomyelin and various sterols.

FIG. 6 is a view showing a thermal-analysis result of the binding activities of GF-Nni for various artificial membranes.

FIG. 7 is a view of a microscopic image showing binding of GF-Nni to a HeLa cell surface.

FIG. 8 is a view of a microscopic image showing binding of GF-Nni to a HeLa cell surface.

FIG. 9 is a view of a microscopic image showing localization of GF-Nni and lysenin in a HeLa cell.

FIG. 10 is a view of a microscopic image showing localization of GF-Nni and BMP in a HeLa cell.

FIG. 11 is a view of an alignment of amino acid sequences of a protein represented by the amino acid sequence of SEQ ID NO: 1 and its homologues.

FIG. 12 is a view showing a result of detecting GF-Nni by electrophoresis, by mixing (i) a protein GF-Nni according to an example of the present invention and (ii) various liposomes and separating this mixture into supernatants (S) and precipitates (P).

FIG. 13 is a graph showing binding activities of GF-Nni for mixtures of various sterols and sphingomyelin.

FIG. 14 is a view of a microscopic image showing a binding site of GF-Nni to a HeLa cell surface.

FIG. 15 is a view of a microscopic image showing distribution of GF-Nni, lysenin, and H-ras in a cell membrane.

FIG. 16 is a view of a microscopic image showing distribution of GF-Nni, lysenin, and K-ras in a cell membrane.

FIG. 17 is a view of a microscopic image showing binding activities of GF-Nni for an intracellular organelle.

FIG. 18 is a view of a microscopic image showing a binding site of GF-Nni within a cell.

FIG. 19 is a view of a microscopic image showing lipid distribution within a cell affected by Niemann-Pick disease.

FIG. 20 is a view of a microscopic image showing lipid distribution on a surface of a cell affected by Niemann-Pick disease.

FIG. 21 is a graph showing effects of GF-Nni on cultured cells (MDCK) infected with an influenza virus.

FIG. 22 is a graph showing effects of GF-Nni on cultured cells (MDCK) infected with an influenza virus.

### DESCRIPTION OF EMBODIMENTS

Described below is a specific description of the present invention.

### Protein

A protein according to the present invention is one being the following (A) or (B):

(A) a protein represented by the amino acid sequence of SEQ ID NO: 1; or
(B) a protein represented by an amino acid sequence in which one or a plurality of amino acid is substituted, deleted, inserted or added in the amino acid sequence of SEQ ID NO: 1, the protein having binding activity specific for a mixture of a sphingolipid and cholesterol.

When used in the present specification, the term “protein” is used exchangeably with “peptide” or “polypeptide”. The protein according to the present invention may be any polypeptide as long as amino acids are bound therein via a peptide bond. However, it is not limited to polypeptides, and the protein can be a composite polypeptide including a non-polypeptide structure. When used in the present specification, the term “non-polypeptide structure” can be, although not limited in particular, a sugar chain, an isoprenoid group, or the like.

The protein represented by the amino acid sequence of SEQ ID NO: 1 has binding activity specific for a mixture of a sphingolipid and cholesterol, as shown in Examples described later. Therefore, the protein according to the present invention can recognize the mixture specifically.

The “mixture of a sphingolipid and cholesterol” in the present specification may be any mixture as long as the mixture contains at least a sphingolipid and cholesterol. The “mixture” denotes a composite in which two or more substances are mixed together, and may also be referred to as a “blend”. Moreover, the “mixture” conceptually encompasses a complex, and the mixture is preferably a complex of a sphingolipid and cholesterol. It is more preferable that the mixture is a lipid raft or a product having an identical composition thereto. Examples of the sphingolipid encompass sphingomyelin or the like. The protein according to the present invention can specifically recognize a mixture of a sphingolipid and cholesterol, thereby being able to specifically detect a mixture of a sphingolipid and cholesterol, more preferably a lipid raft.

The description “having binding activity specific for a mixture of a sphingolipid and cholesterol” means that the protein has binding activity specific for the mixture, but has no binding activity for a sphingolipid alone or cholesterol alone. The expression “have binding activity for” means that the binding activity is higher than that for substances other than the subject to be bound. Moreover, the expression “has no binding activity for sphingolipid alone or cholesterol alone” means that the binding activity is lower than the binding activity for the mixture of the sphingolipid and cholesterol.

The binding activity of the protein may be evaluated by a well-known method in this field. For example, it is possible to employ an ELISA technique (Kiyokawa et al., Biochemistry, 43, 9766 (2004), the entire contents of which are hereby incorporated by reference).

The protein according to the present invention has binding activity specific for a mixture of a sphingolipid and cholesterol, thereby allowing for specifically recognizing the mixture, more preferably a lipid raft. Therefore, by use of the protein according to the present invention, it is possible to detect a lipid raft by a method such as visualization.

Moreover, the protein according to the present invention has binding activity, irrespective of whether the mixture of the sphingolipid and cholesterol takes an ordered liquid phase or a disordered liquid phase, as described in the Examples described later. Namely, the protein according to the present invention recognizes not the physical properties of the mixture but its structure when binding thereto, different from ostreolysin known until now (Sepcic K, Berne S, Rebolj K, Batista U, Plemenitas A, Sentjurc M, Macek P. (2004) Ostreolysin, a pore-forming protein from the oyster mushroom, interacts specifically with membrane cholesterol-rich lipid domains. FEBS Lett. 575 and 81-5., the entire contents of which are hereby incorporated by reference). This hence makes it possible to more specifically recognize the mixture by use of the protein according to the present invention.

The foregoing “one or a plurality of amino acid is substituted, deleted, inserted or added” means that amino acids as many as it can be substituted, deleted, inserted or added by known mutant protein producing methods such as site-directed mutagenesis (1-10, preferably 1-7, more preferably 1-5 amino acids) are substituted, deleted, inserted or added in the amino acid sequence. Such a protein whose amino acid(s) is substituted, deleted, inserted or added may also be called a “mutant protein” or a “mutant.” Mutant protein may be a protein in which mutation is introduced artificially, or may be a mutant protein naturally present and which has been isolated and purified.

A person skilled in the art can easily cause mutation of 1 to 10 amino acids among amino acid residues that constitute a protein, with use of a well-known method. For example, by following the known point-mutation introducing method, it is possible to cause mutation of any base of a polynucleotide which codes for protein. Moreover, by designing a primer corresponding to any site of the polynucleotide that codes for protein, it is possible to produce a deletion mutant or an addition mutant.

The term “mutant”, when used in a description related to protein in the present specification, intends to mean a protein retaining a specific function that a target protein has, i.e. a protein having the function of having binding activity specific for the mixture of a sphingolipid and cholesterol.

It should be noted that it is common knowledge in this field that some amino acids among the amino acid residues that constitute the protein can be easily altered without causing any significant effect on the structure or function of the protein. Furthermore, it is also common knowledge that mutants having no significant change in the structure or function of the protein are present not only in proteins that are artificially altered but also in natural proteins.

Moreover, the protein according to the present invention may be represented by an amino acid sequence that is identical to the amino acid sequence of SEQ ID NO: 1 by at least 70% or more, preferably 80% or more, more preferably 90% or more, and further preferably 95% or more.

Moreover, the protein according to the present invention may be a fused protein to which an additional polypeptide is added. Examples of the additional polypeptide encompass a labeled polypeptide and like polypeptide. The term “labeled polypeptide” indicates a polypeptide used for labeling a protein with use of a known method for example epitope labeling or fluorescence labeling. Examples of the labeled polypeptide encompass epitope-labeled peptide such as His, Myc, and Flag, and fluorescence protein such as GFP, etc. The additional polypeptide may be added to an N terminal or C terminal of the protein according to the present invention. As to the additional polypeptide, it is more preferable that the additional polypeptide is added to the N terminal, in terms of maintaining sufficient binding activity for a mixture. Moreover, the protein according to the present invention may be one whose additional polypeptide is removed from a fused protein after recombination, expression and purification of the fused protein.

The protein according to the present invention may be a protein isolated from a natural supply source, a protein chemically synthesized, or a recombinant protein produced by a gene recombination technique. A method well-known in the field may be employed as a method of obtaining these proteins.

The term “isolated” protein intends to mean a protein obtained from a natural environment. For example, a protein recombined and produced in a host cell is considered as being isolated, as with a natural or a recombinant protein that is substantially purified by any appropriate technique.

A recombinant protein denotes a product produced from a host by a recombination technique. The protein according to the present invention may be glycosylated or may be un-glycosylated, depending on the host used in the recombination production procedure. In some cases, the protein according to the present invention may contain an initial modified methionine residue as a result of performing a host-mediated process. Moreover, it is also possible to employ for example a method that uses a vector and cell (later described), as the recombination production procedure.

The protein according to the present invention may be in a form of a reagent for detecting a lipid raft (reagent for lipid raft detection), which reagent contains the protein. The protein according to the present invention is useful as a reagent for lipid raft detection, since it is possible to specifically recognize a lipid raft as described above. A detailed composition of the reagent for detecting the lipid raft is not limited in particular, and various additives, buffer solutions, or the like may be added in accordance with its use and purpose.

The protein according to the present invention may be a homologue of a protein represented by the amino acid sequence of SEQ ID NO: 1. Examples of the homologue encompass a protein derived from brown beech mushrooms (Hypsizygus marmoreus) (e.g., SEQ ID NO: 13), a protein of Chlamydomonas reinhardtii (EDP07110), a protein of Hordeum vulgare (BAF03218), a protein of Camellia sinensis (ACV60356), a protein of Sorghum bicolor (EER97936), a protein of Zea mays (ACG38107), and a protein of Oryza sativa (EAY93540).

Moreover, a homologue of the protein represented by the amino acid sequence of SEQ ID NO: 1 includes a protein represented by an amino acid sequence in which one or a plurality of amino acid is substituted, deleted, inserted or added in the amino acid sequence of the homologue as described above.

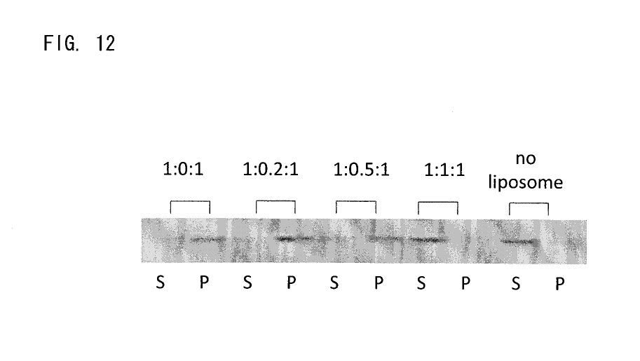

The amino acid sequence of the protein represented by the amino acid sequence of SEQ ID NO: 1 and the amino acid sequence of its homologue were aligned using ClustalW, and were boxshaded using BoxShade. FIG. 11 is a view showing an alignment of the amino acid sequence of the protein represented by the amino acid sequence of SEQ ID NO: 1 and of its homologue. Note that the abbreviations shown in FIG. 11 mean as follows. GF: Grifola frondosa; HM: Hypsizygus marmoreus; CR: Chlamydomonas reinhardtii (EDP07110) HV: Hordeum vulgare (BAF03218), CS: Camellia sinensis (ACV60356), SB: Sorghum bicolor (EER97936), ZM: Zea mays (ACG38107), OS: Oryza sativa (EAY93540).

In FIG. 11, the amino acid sequence marked by black represent an identity of 50% or more, and the amino acid sequence marked by gray is a part representing an identity of 50% or more of a similar amino acid. These results are shown in the lower line as consensus. The capital letters of the alphabet indicate a 100% match.

### Polynucleotide

A polynucleotide according to the present invention can be any polynucleotide as long as it codes for the protein according to the present invention described above. The polynucleotide according to the present invention may be a polynucleotide of the following (A), (B), or (C), for example.

(A) A polynucleotide represented by the base sequence of SEQ ID NO: 2;
(B) A polynucleotide represented by a base sequence in which 1 to 30 bases are substituted, deleted, inserted or added in the base sequence of SEQ ID NO: 2, the polynucleotide coding for a protein having binding activity specific for a mixture of a sphingolipid and cholesterol; or
(C) A polynucleotide that hybridizes under stringent conditions with a polynucleotide consisting of a base sequence complementary to the base sequence of SEQ ID NO: 2, the polynucleotide coding for a protein having binding activity specific for a mixture of a sphingolipid and cholesterol.

The polynucleotide represented by the base sequence of SEQ ID NO: 2 codes for the protein represented by the amino acid sequence of SEQ ID NO: 1.

When used in the present specification, the term “polynucleotide” is used exchangeably with terms “gene”, “DNA”, “RNA”, “nucleic acid”, or “nucleic acid molecule”. When used in the present specification, the term “base sequence” is used exchangeably with terms “nucleic acid sequence” or “nucleotide sequence”, and is recited as a sequence of deoxyribonucleotide (abbreviated as A, G, C, and T), or ribonucleotide (abbreviated to A, G, C, and U).

The polynucleotide according to the present invention can be easily designed by a person skilled in the art, from an amino acid sequence of a protein to be coded for.

The description “one or a plurality of base is substituted, deleted, inserted or added” means that a number of bases is substituted, deleted, inserted or added in the amino acid sequence, which number is a number (1 to 30, preferably 1 to 21, more preferably 1 to 15, further preferably 1 to 5) of bases that can be substituted, deleted, inserted or added, based on known mutant protein producing methods such as site-directed mutagenesis. Such a polynucleotide being substituted, deleted, inserted or added may also be called a “mutant polynucleotide” or a “mutant.” The mutant polynucleotide may be a polynucleotide in which mutation is introduced artificially, or may be a mutant polynucleotide naturally present and which has been isolated and purified.

A person skilled in the art can easily cause mutation of 1 to 30 bases in a base constituting a polynucleotide, with use of a well-known method. For example, by following the known point-mutation introducing method, it is possible to cause mutation of any base of a polynucleotide. Moreover, by designing a primer corresponding to any site of the polynucleotide, it is possible to produce a deletion mutant or an addition mutant.

The term “mutant”, when used in a description related to polynucleotide in the present specification, intends to mean a polynucleotide that codes for a protein retaining a specific function that a target protein has, i.e. a protein having a function of having binding activity specific for the mixture of sphingolipid and cholesterol.

The polynucleotide according to the present invention is a polynucleotide that hybridizes under stringent conditions with a polynucleotide consisting of a base sequence complementary to the base sequence of SEQ ID NO: 2, and may be a protein having binding activity specific for a mixture of a sphingolipid and cholesterol.

The hybridization may be performed by a well-known method such as the method disclosed in Sambrook et al., Molecular Cloning, A Laboratory Manual, 2d Ed., Cold Spring Harbor Laboratory (1989), the entire contents of which are hereby incorporated by reference. Usually, the stringency increases (it becomes difficult to hybridize) as the temperature increases and as the salt concentration decreases, thereby allowing for obtaining a more homologous polynucleotide. A suitable hybridization temperature differs depending on the base sequence and the length of that base sequence. For example, when a DNA fragment constituted of 18 bases is used as a probe, which DNA fragment codes for six amino acids, it is preferable that the temperature be not more than 50° C.

The term “stringent conditions” in the present specification intends to mean (i) incubating the polynucleotide in a hybridization solution (containing 50% formamide, 5×SSC (150 mM of NaCl, 15 mM of trisodium citrate), 50 mM of sodium phosphate (pH 7.6), 5× Denhardt's solution, 10% dextran sulfate, and 20 μg/ml denatured shear salmon sperm DNA) overnight at 42° C., and thereafter (ii) washing, at about 65° C., a filter in 0.1×SSC.

Moreover, the polynucleotide according to the present invention may be represented by a base sequence homologous to the base sequence of SEQ ID NO: 2 by 70% or more, preferably 80% or more, more preferably 90% or more, further preferably 95% or more.

The polynucleotide according to the present invention encompasses not only double strand DNAs but also single strand DNAs of a sense strand and an antisense strand constituting the double strand DNA, and RNA. The antisense strand can be used as a probe or as an antisense drug. Moreover, DNA encompasses cDNA, genomic DNA, etc. which are obtained by cloning, chemosynthesis technology, or a combination of those. Furthermore, the polynucleotide according to the present invention may include sequences such as a sequence of an untranslated region (UTR) or a sequence of a vector sequence (including expression vector sequence).

An example of a method of obtaining the polynucleotide according to the present invention is a method of isolating a DNA fragment containing the polynucleotide according to the present invention and carrying out cloning thereto, by a known technique. For example, a probe that specifically hybridizes with a part of a base sequence of the polynucleotide of the present invention can be prepared, to screen a genomic DNA library, a cDNA library, etc. The probe used as such may be of any sequence and/or length as long as it is a probe that specifically hybridizes with at least a part of a base sequence of the polynucleotide according to the present invention or its complementary sequence.

Alternatively, another example of a method of obtaining the polynucleotide according to the present invention is a method using an amplification technique such as PCR. For example, a primer is prepared among sequences (or their complementary sequences) on the 5′-end and the 3′-end of cDNA(s) of the polynucleotide of the present invention, to perform PCR using these primers by having a genomic DNA, cDNA or the like serve as templates. By amplifying a DNA region sandwiched between the primers, it is possible to obtain a large amount of DNA fragments that contain the polynucleotide according to the present invention.

Moreover, it is preferable that the polynucleotide according to the present invention is obtained from basidiomycetes, and is more preferable that the polynucleotide be obtained from a Grifola genus. For example, the polynucleotide represented by the base sequence of SEQ ID NO: 2 is obtainable from the genomic DNA, cDNA, or the like of Grifola frondosa, by the method described above.

### Vector

The vector according to the present invention may be any vector as long as it contains the polynucleotide described above, and for example can be an expression vector for expressing the protein according to the present invention. The vector according to the present invention may be a vector used for in vitro translation or may be a vector used for recombinant expression.

The vector according to the present invention may be, for example, a recombinant expression vector into which a cDNA of a polynucleotide coding for the protein according to the present invention is inserted. Examples of a method of preparing the recombinant expression vector include methods using for example plasmid, phage, or cosmid. However, how the recombinant expression vector is prepared is not limited in particular. Moreover, it is also possible to employ a known method for preparing the vector.

The vector is not limited in particular as to its specific kind, and a kind of vector that can be expressed in a host cell is selected as appropriate. Namely, a kind of vector for positively expressing the polynucleotide according to the present invention is selected in accordance with a kind of a host cell; the polynucleotide of the present invention is incorporated into the vector, to be used as an expression vector. Moreover, it is preferable that the vector according to the present invention includes a promoter sequence for making the protein coded by the polynucleotide contained in a vector expressed. The promoter sequence is preferably selected as appropriate in accordance with the kind of host cell. As such, the vector according to the present invention may be a vector in which the polynucleotide according to the present invention and a promoter sequence selected as appropriate are incorporated into various plasmids or the like.

The host cell is not limited in particular, and various conventionally known cells can be suitably used. For example, the host cell can be a procaryote host or an eukaryote host, and specifically, examples thereof encompass bacterial cells such as Escherichia coli, yeast cells such as budding yeast (Saccharomyces cerevisiae) and fission yeast (Schizosaccharomyces pombe), a higher plant cell, an insect cell, and a mammalian cell.

How the vector is introduced into a host cell, i.e., a transformation method, is not limited in particular. It is possible to suitably use conventionally known procedures, such as electroporation, calcium phosphate transfection, a liposome method, and a DEAE dextran process.

### Transformant

A transformant according to the present invention is a transformant into which a polynucleotide coding for the protein according to the present invention described above is introduced. The term “transformant” in the embodiment is a concept encompassing not only a cell, a tissue, or an organ, but also a living organism.

It is preferable that the transformant according to the present invention has the polynucleotide according to the present invention introduced in a host cell in a manner allowing expression therein.

An example of how to produce the transformant according to the present invention is a method of introducing the vector according to the present invention into a host cell. The host cells described above can be used as the host cells. Moreover, the transformant according to the present invention can be cultured or grown by a conventionally known method.

Use of the transformant according to the present invention allows for producing the protein according to the present invention within the transformant; this makes it possible to easily carry out bulk preparation of the protein.

### Antibody

An antibody according to the present invention is an antibody obtained by a known method as a polyclonal antibody or a monoclonal antibody, with use of, as an antigen, (i) the protein according to the present invention described above or (ii) its partial peptide. The known method may be, for example, the method disclosed in the following literatures (Harlow et al., “Antibodies: A laboratory manual” (Cold Spring Harbor Laboratory, New York (1988)), and Iwasaki et al., “Tankuro-n Koutai Haiburido-ma to ELISA (Monoclonal Antibody, Hybridoma and ELISA)”, Kodansha (1991)). Note that the entire contents of these literatures are hereby incorporated by reference. An antibody prepared as such is effective in detection of the protein of the present invention.

### Use of Protein According to the Present Invention

The protein according to the present invention can be suitably used in a method of detecting a mixture of a sphingolipid and cholesterol.

A method of detecting a mixture of a sphingolipid and cholesterol according to the present invention uses the protein according to the present invention described above. For example, as a method of detecting the mixture in an object to be examined, a method including the following steps may be employed. Note that the mixture may be a lipid raft.

1) The step of providing a protein according to the present invention to a subject to be examined;
2) The step of detecting whether specific binding with the protein according to the present invention is present or not; and
3) The step of determining, wherein, when the specific binding is present, it is determined that a mixture of a sphingolipid and cholesterol is present in the subject to be examined.

Conventionally publicly known steps may be used as these steps. For example, in the step 2), whether the specific binding is present or not can be detected by (i) removing the protein according to the present invention that did not specifically bind in the step 1), and (ii) detecting the remaining protein. Examples of methods that can be employed for detecting the protein include conventionally publicly known methods, such as (a) visualizing the protein by applying a fluorescent protein and causing the protein to emit color or light, and (b) detecting the protein by ELISA. The visualized protein can be observed by a fluorescence microscope or the like.

By use of the method described above, it is possible to detect localization of a lipid raft in an organism sample (a cell, tissue, etc.), for example. Therefore, the present invention is useful in studies of kinetics, function, and the like of a lipid raft.

For example, the present invention can be used for detailed analysis, diagnosis, and the like of a disease associated with a lipid raft. Lipid metabolism disorder is an example of a disease associated with a lipid raft. One example of a lipid metabolism disorder is the Niemann-Pick disease. For example, by studying a localization of the protein according to the present invention, it is possible to analyze the localization of the lipid raft in the cell affected by the disease described above, and diagnose as to whether a person suffers from the disease described above.

Moreover, since the protein according to the present invention can specifically bind to the mixture of a sphingolipid and cholesterol, it is possible to use the protein according to the present invention in a method of purifying the mixture, preferably a method of purifying a lipid raft. As the method of purifying the mixture, various conventionally known purification methods that utilize a specific binding between substances are applicable.

Moreover, since the protein according to the present invention can bind specifically to a lipid raft, it is possible to suitably use the protein in medicament for various diseases that associate with the lipid raft. One example of the diseases associated with the lipid raft is viral infections. For example, the AIDS virus is known as being covered with the lipid raft. Moreover, as described in Examples later described, the protein according to the present invention serves to prevent viral infection of a cell, particularly infection with influenza viruses. Therefore, the protein according to the present invention can be used for medicament for preventing viral infections.

The target viruses that can be prevented from infection with use of the present invention is not limited to the AIDS virus and the influenza viruses described above, and any virus that targets the lipid raft may be prevented (for example, a hepatitis C virus).

Moreover, it is possible to treat viral infections by administering the protein according to the present invention. The present invention also provides a treatment method of viral infections including administering to a target object the protein according to the present invention.

Moreover, since the protein according to the present invention can inhibit viral infections to a cell as described above, the protein can be used as a virus infection inhibitor. What is only required in a virus infection inhibitor according to the present invention is to contain the protein according to the present invention in the virus infection inhibitor. The virus may be, for example, influenza viruses or the like.

Moreover, the protein according to the present invention has very low toxicity. Hence, it is possible to suitably use the protein in medicament and the like as described above.

### Lipid Raft Detection Kit

A kit for lipid raft detection according to the present invention (hereinafter, also referred simply as “kit”) is a kit for detecting a lipid raft. The kit includes at least any one of the followings selected from the group consisting of: a protein, a polynucleotide, a vector, a transformant, and an antibody, each of which are described above.

By including the protein described above, the kit according to the present invention can detect the mixture of a sphingolipid and cholesterol, in particular a lipid raft, by use of the protein. Note that the protein is preferably a protein to which a labeled polypeptide is added. Moreover, the polynucleotide, the vector, and the transformant each described above can be used to obtain the protein described above. Furthermore, the antibody described above is useful for detecting the protein described above.

The kit according to the present invention can be used for detecting, for example, the mixture of the sphingolipid and cholesterol described above. Hence, the kit can be suitably used to test an organism sample as to a lipid raft, such as whether or not a lipid raft is present therein and a localization of the lipid raft therein.

It is preferable that the kit according to the present invention further includes a substance having binding activity specific for sphingomyelin. Examples of such a substance encompass lysenin and a protein derived from lysenin. It is possible to preferably use lysenin and a protein derived from lysenin, which are disclosed in Patent Literatures 1 and 2 (as to Patent Literatures 1 and 2, see specifications of corresponding United States Patent Publication No. 10/138634 A and U.S. Ser. No. 11/223,974 A, respectively, the entire contents of which are hereby incorporated by reference). This allows for comparison of the localization of the mixture of sphingomyelin and cholesterol, for example, a lipid raft, with the localization of sphingomyelin, thereby allowing for accurately studying the localization, kinetics, and the like of the lipid raft.

Moreover, the kit according to the present invention can further include a polynucleotide coding for lysenin or a protein derived from lysenin, a vector containing the polynucleotide, a transformant into which the polynucleotide or the vector is introduced, an antibody against lysenin or the protein derived from lysenin, etc.

Moreover, it is preferable that the kit according to the present invention further includes a substance having binding activity specific for cholesterol. Examples of such a substance encompass, for example, polyethylene glycol cholesterylether and polyethylene glycol 2-aminoethyl cholesterylether. Moreover, the substance can be in the form of a cholesterol detecting reagent. As the substance and the cholesterol detecting reagent containing the substance, it is possible to suitably use those disclosed in Patent Literature 3 and Patent Application 4 (as to Patent Application 4, see specification of corresponding Publication U.S. patent application Ser. No. 10/516,072, the entire contents of which are hereby incorporated by reference). This allows for comparison of a localization of the mixture of sphingomyelin and cholesterol, for example, the lipid raft, with the localization of cholesterol. As a result, it is possible to more accurately study the localization, kinetics, and the like of the lipid raft.

It is preferable that the kit according to the present invention includes both a substance having binding activity specific for sphingomyelin described above and a substance having binding activity specific for cholesterol. This makes it possible to compare the localization of the mixture of sphingomyelin and cholesterol, for example, a lipid raft, with the localization of sphingomyelin and with the localization of cholesterol. As a result, it is possible to further thoroughly study on the localization, kinetics and the like of the lipid raft.

### Additional Matters

It should be noted that the protein according to the present invention preferably has a labeled polypeptide added thereto.

Moreover, it is preferable that the kit for detecting a lipid raft according to the present invention further includes at least one of (i) a substance having binding activity specific for sphingomyelin and (ii) a substance having binding activity specific for cholesterol.

The present invention is not limited to the embodiments described above, and various modifications may be made within the scope of the claims. Namely, embodiments accomplished by combining technical measures varied as appropriate within the scope of the claims are included within the technical scope of the present invention.

### Examples

The following description raises Examples to describe the present invention in further details. Note however that the present invention is not limited to these Examples.

### Example 1

### Identification of Novel Protein and Gene

The inventors of the present invention identified a novel protein GF-Nni (SEQ ID NO: 1) from Hen-of-the-woods (Grifola frondosa), which novel protein specifically binds to a mixture of a sphingolipid and cholesterol. Specifically, a protein that binds to sphingomyelin (obtained from Avanti)/cholesterol (obtained from Sigma) (1:1) liposome was purified from a sap of Hen-of-the-woods protein, to obtain partial sequences of an amino acid (MLYGVEIDEQYLRVMEEYKDKEVITQADMAKVALQRKNVYQ DQAEKRQAELKAEYGVGV, XHLLRVYATX, HGQTGNETTAVEY, SFRGHFGAHTREK, TTAYVEYVYSR).

Next, the inventors identified a base sequence (SEQ ID NO: 2) of a gene coding for the protein GF-Nni. More specifically, as a result of conducting tblastn analysis on the obtained partial sequences, on the basis of a cDNA database of Hen-of-the-woods managed by Yukiguni Maitake Co., Ltd., the inventors found a DNA sequence of 373 bp in the registered sequences. Based on this sequence, two primers for RACE (5′-RACE-GF (SEQ ID NO: 3) and 3′-RACE-GF (SEQ ID NO: 4)) were designed; by performing RACE-PCR using a SMARTer™ RACE cDNA Amplification Kit (Takara Bio, Inc.), an upstream sequence and a downstream sequence of a gene that codes for GF-Nni were determined.

Next, the gene represented by the base sequence of SEQ ID NO: 2 obtained from Hen-of-the-woods (Grifola frondosa) was cloned. More specifically, a forward primer Nni-F (SEQ ID NO: 5) and a reverse primer Nni-R (SEQ ID NO: 6), each for cloning, were designed from the determined GF-Nni sequence, and a full-length gene of GF-Nni was cloned by the PCR technique. The full length of GF-Nni was inserted into pENTR/SD/D-TOPO vector of Invitrogen Corporation.

With use of this gene, the protein GF-Nni was expressed and purified by a gene recombination technique. More specifically, the GF-Nni gene was introduced into a pET-28b vector, to express the protein by Escherichia coli BL21 (DE3). Thereafter, the protein was purified using a nickel column.

### Example 2

With use of protein GF-Nni purified from the Hen-of-the-woods, bindings thereof to liposomes constituted of various lipids and cholesterol (artificial membrane) (1:1) were studied. Used as the lipids of the artificial membrane were sphingomyelin (SM), phosphatidylcholine (PC), phosphatidylethanolamine (PE), phosphatidylserine (PS), phosphatidylinositol (PI), and phosphatidylglycerol (PG). These lipids were purchased from Avanti. Cholesterol was purchased from Sigma.

First, GF-Nni and liposome were mixed together, and this mixture was then separated into supernatants (S) and precipitates (P) (which contains the liposome) by centrifugation. Next, these supernatants (S) and precipitates (P) were subjected to electrophoresis, respectively, and the protein was stained by a known method. In this way, which fraction contained G-Nni was examined.

FIG. 1 is a view showing a result of detecting GF-Nni by electrophoresis by mixing (i) a protein GF-Nni according to an example of the present invention and (ii) various liposomes and separating this mixture into supernatants (S) and precipitates (P). As shown in FIG. 1, GF-Nni was detected in a precipitate fraction just when a liposome made of sphingomyelin and cholesterol was used. This result suggested that the protein according to the present invention specifically binds to a mixture of sphingomyelin and cholesterol.

### Example 3

Next, with use of the purified protein GF-Nni, binding activity thereof for mixtures of various lipids and cholesterol (1:1) were further observed. Used as the lipids, besides those used in Example 2, were galactosylceramide (GalCer) (obtained from Avanti), glucosylceramide (GlcCer) (obtained from Matreya), lactosylceramide (LacCer) (obtained from Avanti), sphingosillphosphorylcholine (SPC) (obtained from BioMol), ganglioside (GM1) (obtained from Wako Pure Chemicals), and ceramide (Cer) (obtained from Avanti).

First, ethanol solutions containing various lipids and cholesterol at a ratio of 1:1 were provided into a 96-well plastic plate, and then ethanol was evaporated. To this, GF-Nni, an anti-GF-Nni antibody (rabbit polyclonal antibody), and a HRP-conjugated anti-rabbit antibody were added in this order. Thereafter, the amounts of GF-Nni that bound to the mixtures of the lipid and cholesterol were measured by the known ELISA technique (solid phase technique) (see Kiyokawa E, Makino A, Ishii K, Otsuka N, Yamaji-Hasegawa A, Kobayashi T. (2004) Recognition of sphingomyelin by lysenin and lysenin-related proteins. Biochemistry 43, 9766-9773, the entire contents of which are hereby incorporated by reference).

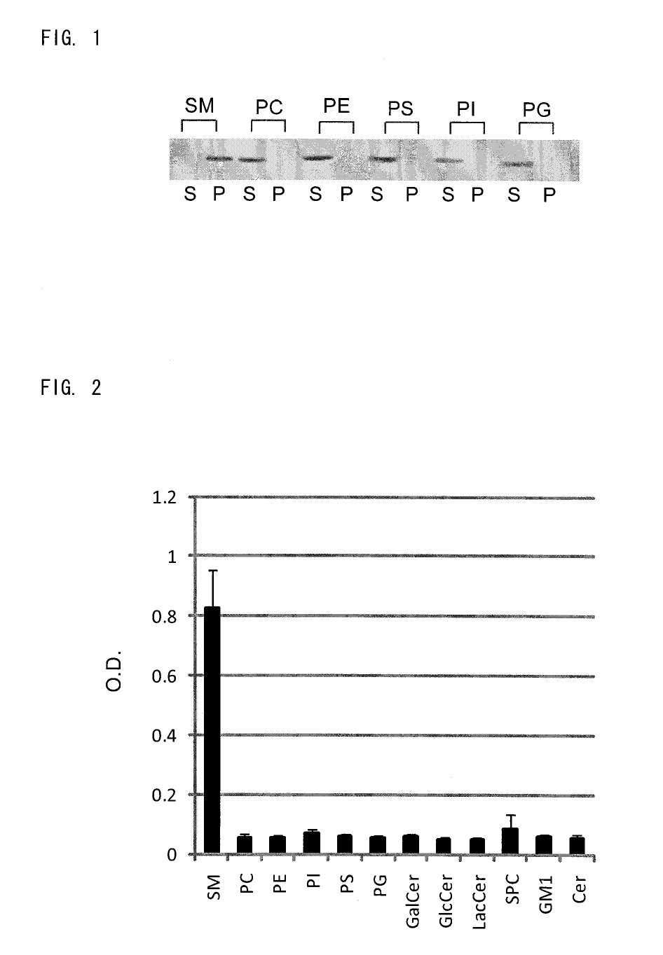

FIG. 2 is a graph showing binding activities of GF-Nni for the mixtures of the lipids and cholesterol. As shown in FIG. 2, GF-Nni showed high binding activity only for the mixture of sphingomyelin and cholesterol. This result demonstrated that the protein according to the present invention selectively bound to the mixture of sphingomyelin and cholesterol.

### Example 4

Binding activities of the purified protein GF-Nni for a mixture in which sphingomyelin and cholesterol were contained in different ratios were observed with use of the purified protein GF-Nni, by the ELISA technique described above.

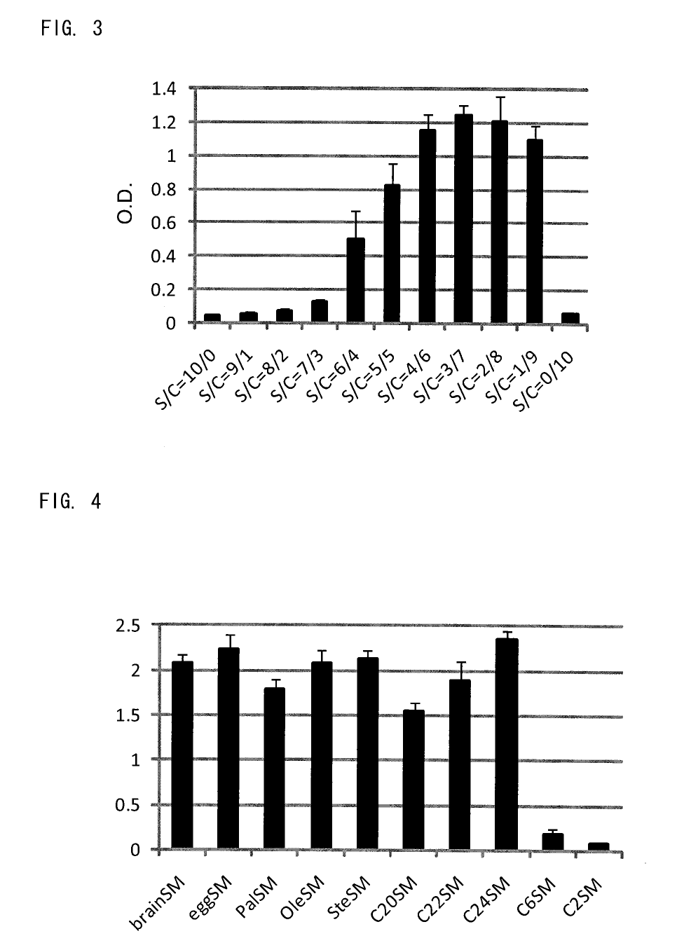

FIG. 3 is a graph showing binding activities of GF-Nni for the mixture of sphingomyelin (S) and cholesterol (C). As shown in FIG. 3, the binding activities of GF-Nni for the mixture of sphingomyelin and cholesterol was dependent on a ratio (S/C) in which sphingomyelin and cholesterol were contained in the mixture.

This result demonstrated that GF-Nni binds efficiently to the mixture that had a ratio of sphingomyelin/cholesterol in a range of 6/4 to 1/9. Moreover, the results showed that the binding activity of GF-Nni was very low for a sample containing only sphingomyelin (S/C=10/0) and to a sample containing only cholesterol (S/C=0/10).

As from the above, it was strongly suggested that the protein according to the present invention specifically binds to the mixture of sphingomyelin and cholesterol. Moreover, it was suggested that the protein according to the present invention has high binding activities for a mixture at least whose ratio of sphingomyelin to cholesterol is 6/4 to 1/9.

### Example 5

With use of the protein GF-Nni that was expressed and purified by the same method as Example 1, binding activities for mixtures of various sphingomyelins and cholesterol (1:1) were studied by the ELISA technique described above. Used as the sphingomyelins were swine brain-derived sphingomyelin (brainSM) (obtained from Avanti), egg yolk-derived sphingomyelin (EggSM) (obtained from Avanti), synthetic sphingomyelin (PalSM) containing palmitic acid (C16:0) (obtained from Avanti), synthetic sphingomyelin containing oleic acid (C18:1) (OleSM) (obtained from Sigma), synthetic sphingomyelin containing stearic acid (C18:0) (SteSM) (obtained from Matreya), and synthetic sphingomyelins containing a saturated fatty acid of a carbon number of 20, 22, 24, 6, or 2 (C20SM, C22SM, C24SM, C6SM, C2SM, respectively) (obtained from Matreya).

FIG. 4 is a graph showing binding activities of GF-Nni for the mixtures of various sphingomyelins and cholesterol. As shown in FIG. 4, GF-Nni showed high binding activity for any of swine brain-derived sphingomyelin and egg yolk-derived sphingomyelin. Moreover, GF-Nni bound to the mixtures that contain sphingomyelin having a fatty acid of a carbon number of 18 or more, irrespective of whether the fatty acid was saturated fatty acid or unsaturated fatty acid.

In a case in which the mixture of sphingomyelin and cholesterol includes sphingomyelin having a saturated fatty acid as its fatty acid, the mixture is of an ordered liquid phase, whereas in a case in which the sphingomyelin has an unsaturated fatty acid, the mixture is of a disordered liquid phase. The ordered liquid phase is a state in which orientation of the fatty acid (hydrocarbon chain) of sphingomyelin is uniform, and the disordered liquid phase indicates a state in which the orientation of the fatty acid is in disorder.

The result of the Examples demonstrated that the protein according to the present invention has binding activity irrespective of whether the mixture serving as the object to be bound is in the ordered liquid phase or in the disordered liquid phase. That is, the result demonstrated that the protein according to the present invention binds to the mixture not by recognizing the physical properties of the mixture but by recognizing the structure of the mixture.

Moreover, the result demonstrated that the protein according to the present invention has binding activity for mixtures containing sphingomyelin having a long fatty acid of a carbon number of at least 7, in particular sphingomyelin having a fatty acid of a carbon number of 18 or more. Moreover, the result demonstrated that the protein according to the present invention can bind to mixtures containing sphingomyelin of various origins.

### Example 6

With use of the protein GF-Nni expressed and purified by the same method as Example 1, binding activities for mixtures of sphingomyelins and various sterols (1:1) were studied by the ELISA technique described above. Used as the sterols were cholesterol (cholesterol), ergosterol (ergosterol), lanosterol (lanosterol), coprostane (coprostane), cholesteryl acetate (chol acetate), 5α-chol (5a chol), 5α-chol-3β-one (5a-chol-3b-one), 5α-chol-7-en-3b-ol (5α-chol-7-en-3b-ol), 5α-chol-3β-ol (5α-chol-3b-ol), and 5β-chol-3a-ol (5b-chol-3a-ol).

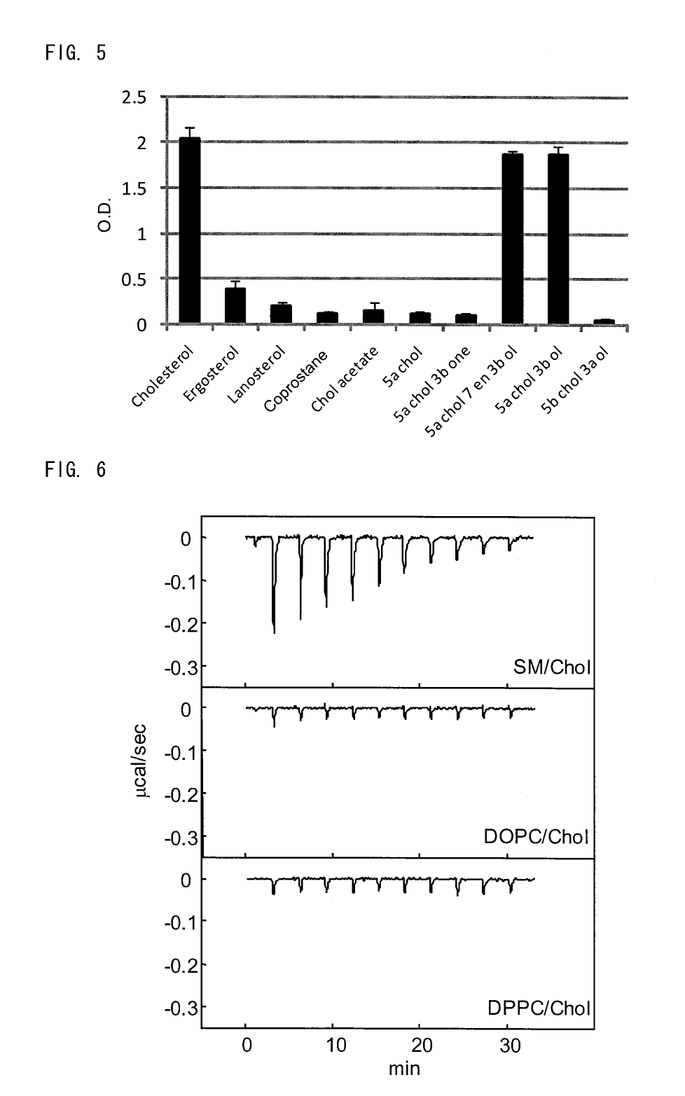

FIG. 5 is a graph showing binding activities of GF-Nni for the mixtures of sphingomyelin and various sterols. As shown in FIG. 5, the result demonstrated that GF-Nni recognizes differences between sterol structures.

### Example 7

The protein GF-Nni expressed and purified in the same method as Example 1 was subjected to thermal analysis with use of a differential thermal analyzer (see Ishitsuka R, Yamaji-Hasegawa A, Makino A, Hirabayashi Y, Kobayashi T. (2004) A lipid-specific toxin reveals heterogeneity of sphingomyelin-containing membranes. Biophys. J. 86 and 296-307., the entire contents of which are hereby incorporated by reference), to observe binding activities for various artificial membranes. Used as the artificial membranes were an artificial membrane of sphingomyelin (obtained from Matreya) and cholesterol (obtained from Sigma) (SM/chol) (1:1), an artificial membrane of DOPC (1,2-dioleoyl-sn-glycero-3-phosphatidylcholine) (obtained from Avanti) and cholesterol (DOPC/chol) (1:1), and an artificial membrane of DPPC (dipalmitoyl phosphatidyl choline) (obtained from Avanti) and cholesterol (DPPC/chol) (1:1).

FIG. 6 shows a thermal analysis result showing the binding activities of GF-Nni for various artificial membranes. As shown in FIG. 6, although GF-Nni bound to SM/chol, GF-Nni did not bind to DOPC/chol and DPPC/chol. GF-Nni did not bind to DPPC/chol that takes an ordered liquid phase. This explains that GF-Nni recognizes not the physical properties but the structure of a subject to be bound to.

### Example 8

Binding abilities of the purified protein GF-Nni to a cell surface were studied.

First, the following cells were fixed: (i) untreated HeLa cells (control), (ii) HeLa cells that had been treated with sphingomyelinase (SMase) to eliminate sphingomyelin, and (iii) HeLa cells that had been treated with methyl beta cyclodextrin (MbetaCD) to eliminate cholesterol. Next, after binding GF-Nni to the surfaces of these HeLa cells, this GF-Nni was visualized with use of an anti-GF-Nni antibody and an Alexa 544-labeled anti-rabbit antibody.

Moreover, for comparison, (i) lysenin that recognizes sphingomyelin and (ii) a cholesterol-binding domain (D4) of θ-toxin that recognizes cholesterol were similarly bound to the HeLa cell as described above, and then were visualized using the fluorescent antibody.

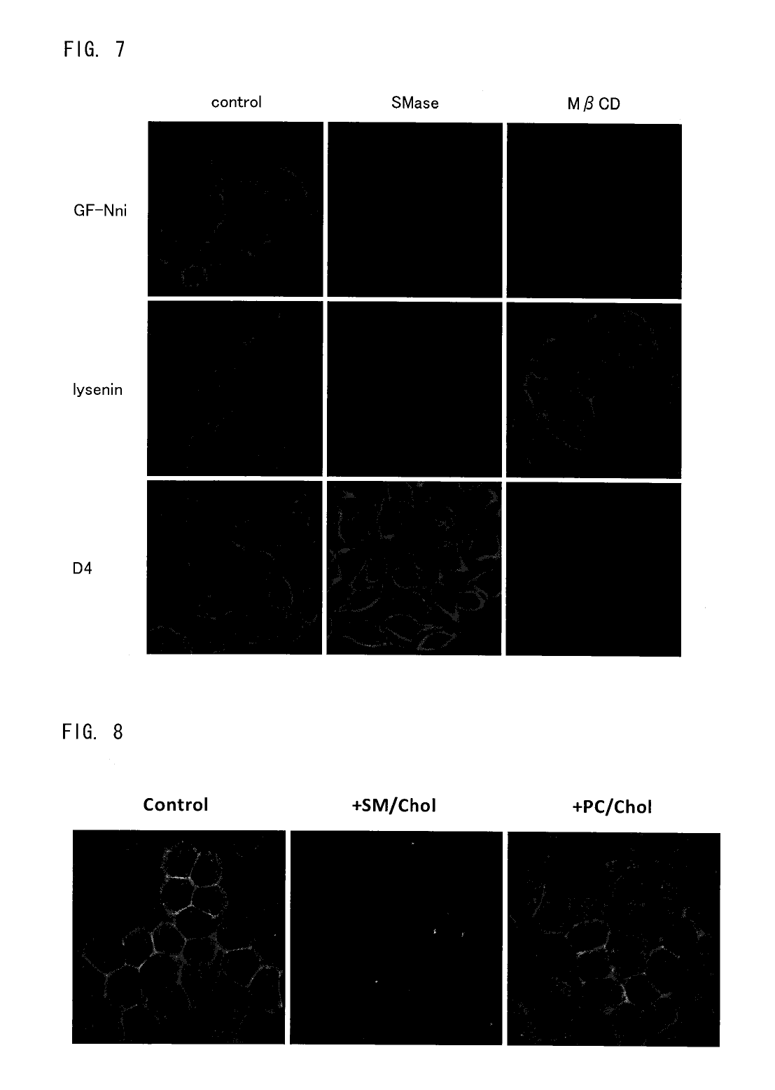

FIG. 7 is a view of microscopic images illustrating the binding of GF-Nni onto the surface of a HeLa cell. As shown in FIG. 7, although GF-Nni binds to the surface of an untreated HeLa cell, GF-Nni does not bind to a cell that had been treated with SMase and to a cell from which cholesterol had been eliminated. On the other hand, lysenin bound to the cell from which cholesterol had been eliminated, and D4 bound to the cell that had been treated with SMase.

### Example 9

Binding abilities of GFP-labeled GF-Nni to a cell was observed, under a condition in which various artificial membranes coexist.

A gene (SEQ ID NO: 8) that codes for a protein GFP-GF-Nni (SEQ ID NO: 7), in which GFP is fused to the N terminal of GF-Nni, was produced. With use of this gene, a GFP-labeled GF-Nni was expressed and purified, by a gene recombination technique. More specifically, a gene that codes for EGFP and a gene that codes for GF-Nni were introduced into a pET-28b vector, to express GFP-GF-Nni with use of Escherichia coli BL21 (DE3) into which the vector was introduced. Thereafter, GFP-GF-Nni was purified with use of a nickel column.

Binding abilities of the purified protein to the surface of the HeLa cell were studied (i) in a condition in which no artificial membrane coexists (control), (ii) in the presence of an artificial membrane of sphingomyelin/cholesterol (SM/Chol) (1:1), and (iii) in the presence of an artificial membrane of phosphatidylcholine/cholesterol (PC/Chol) (1:1).

FIG. 8 is a view of microscopic images showing the binding of GF-Nni to the surface of a HeLa cell. As shown in FIG. 8, GF-Nni binds to the HeLa cell surface under the condition in which no artificial membrane is present. Although this binding was inhibited in the presence of SM/Chol, the binding was not inhibited in the presence of PC/Chol. From this result, support was provided for the fact that GF-Nni binds to a surface of a cell via the mixture of sphingomyelin and cholesterol.

### Example 10

Localizations of GF-Nni and lysenin in a HeLa cell were observed. Specifically, after fixing a cell, the cell was frozen and thawed to make its membrane permeable. Thereafter, GF-Nni and lysenin were added to the cell, to visualize intracellular localizations of GF-Nni and lysenin with use of respective antibodies against the proteins.

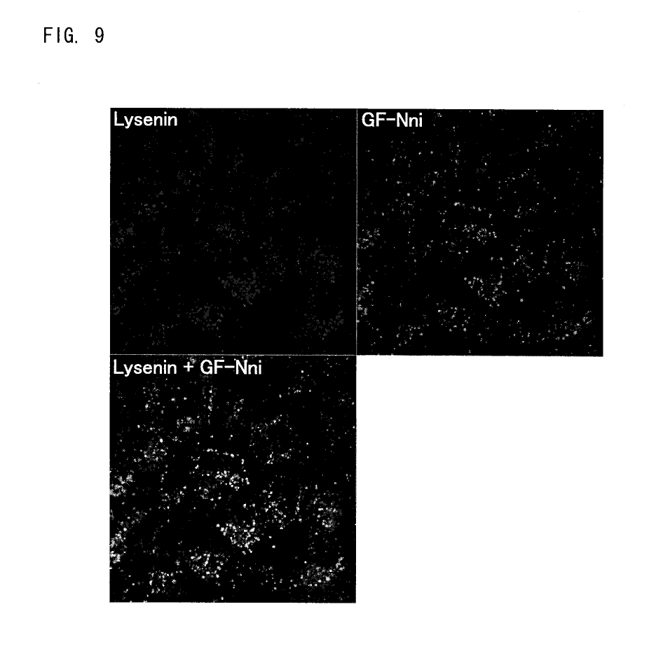

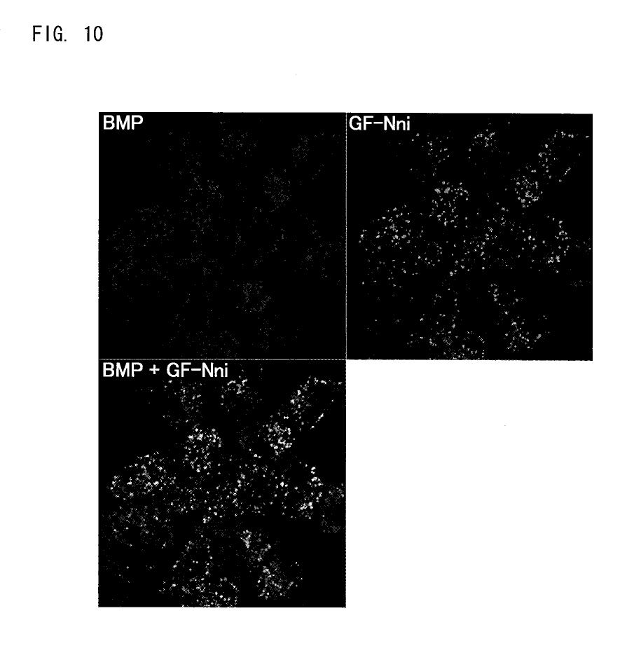

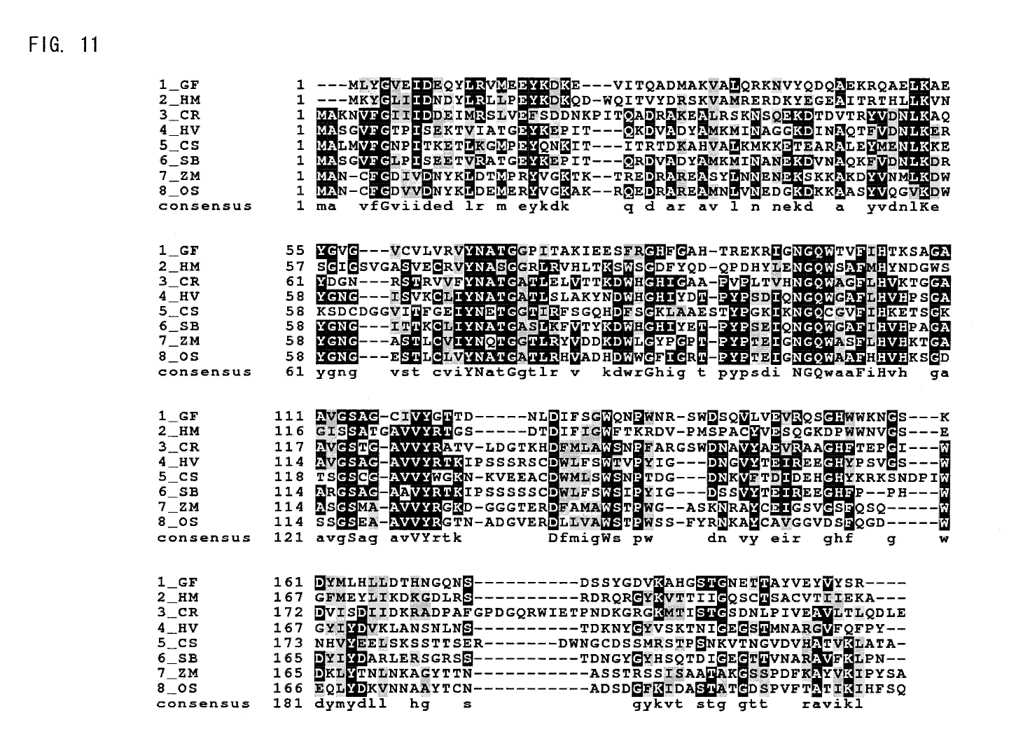

FIG. 9 is a view of microscopic images illustrating the localizations of GF-Nni and lysenin within a HeLa cell. Specifically, FIG. 9 is a view including (i) an image (GF-Nni) indicating the localization of GF-Nni, (ii) an image (Lysenin) indicating the localization of lysenin, and (iii) an image (Lysenin+GF-Nni) in which the images of (i) and (ii) are merged. The localizations of GF-Nni and lysenin match each other, thereby clarifying that the localization of the mixture of sphingomyelin and cholesterol to which GF-Nni binds matches the localization of the sphingomyelin to which lysenin binds.

Thus, by use of the protein according to the present invention, it is possible to easily detect the localization of the mixture (lipid raft) of sphingomyelin and cholesterol within the cell. Moreover, use of the protein according to the present invention in combination with lysenin allows for comparison of the localization, kinetics, and the like between a lipid raft and sphingomyelin that has not formed the lipid raft.

### Example 11

Localization of GF-Nni in a HeLa cell and that of a late endosome marker BMP were observed. Specifically, after fixing a cell, the cell was frozen and thawed to make its membrane permeable. Thereafter, GF-Nni and an antibody against BMP (an endosome-specific lipid) were added to the cell, to visualize the GF-Nni and the BMP by the fluorescent antibody method for observing the localizations thereof by microscopic observation.

FIG. 10 is a view of a microscopic image showing the localization of GF-Nni and BMP in a HeLa cell. Specifically, FIG. 10 is a view including (i) an image (GF-Nni) indicating the localization of GF-Nni, (ii) an image (BMP) indicating the localization of BMP, and (iii) an image (BMP+GF-Nni) in which the images of (i) and (ii) are merged. It was made clear from FIG. 10 that the localization of the mixture of sphingomyelin and cholesterol to which the GF-Nni binds and the localization of the late endosome partially matches.

As described above, the use of the protein according to the present invention allows for comparison of (i) the localization of the mixture of sphingomyelin and cholesterol (lipid raft) within the cell with (ii) the localization of other organelles. This as a result makes it possible to predict kinetics, functions and the like of a lipid raft within the cell.

As described above, the present invention is useful for studying the localizations, kinetics, functions and the like of a lipid raft within the cell.

### Example 12

As one example of the present invention, the inventors found a gene that codes for a homologue protein HM-Nni of the brown beech mushrooms (Hypsizygus marmoreus) by the following method, which protein HM-Nni is homologous to the protein GF-Nni. Namely, on the basis of the amino acid sequence of GF-Nni, tblastn analysis was performed to a cDNA database of brown beech mushrooms managed by Yukiguni Maitake Co., Ltd., to obtain a DNA fragment 228 bp having 36% homology in amino acid. Based on this sequence, two RACE primers (5′-RACE-HM (SEQ ID NO: 9) and 3′-RACE-HM (SEQ ID NO: 10)) were designed, and an unknown upstream sequence and downstream sequence were determined by a RACE-PCR technique using the SMARTer™ RACE cDNA Amplification Kit (Takara Bio, Inc.) (SEQ ID NO: 14). From the determined HM-Nni sequence, a forward primer HM-F (SEQ ID NO: 11) and a reverse primer HM-R (SEQ ID NO: 12), each for cloning, were designed, to clone a full-length gene of HM-Nni by PCR. With use of this gene, the protein HM-Nni (SEQ ID NO: 13) was expressed and purified by the same method as Example 1.

### Example 13

Binding of GF-Nni to a liposome was studied by the same method as Example 2, with use of the protein GF-Nni that was expressed and purified in the same method as Example 1. Used as the liposome was a sphingomyelin/phosphatidylcholine/cholesterol liposome (1:0:1, 1:0.2:1, 1:0.5:1, 1:1:1).

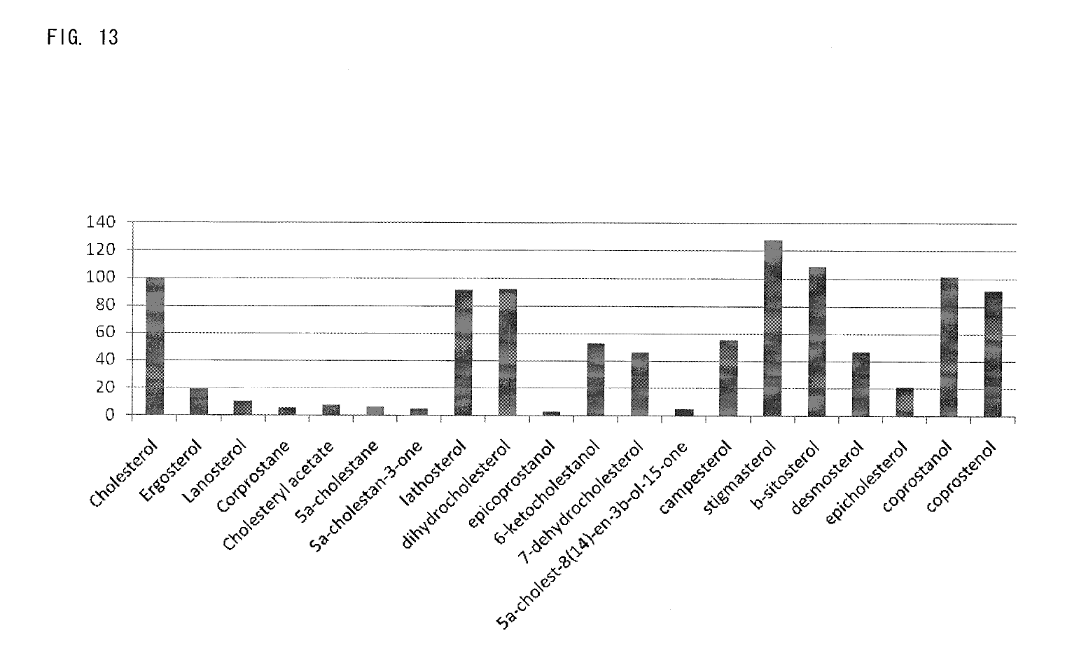

FIG. 12 is a view showing a result of detecting GF-Nni by electrophoresis by mixing (i) a protein GF-Nni according to an example of the present invention and (ii) various liposomes and separating this mixture into supernatants (S) and precipitates (P).

As shown in FIG. 12, the more a ratio of phosphatidylcholine in the liposome increased, the more the binding of GF-Nni was inhibited. This result demonstrated that although GF-Nni was not capable of forming a complex with sphingomyelin and cholesterol, GF-Nni was capable of binding to a sphingomyelin/cholesterol complex that was present in advance.

### Example 14

Binding activities of the protein GF-Nni expressed and purified in the same method as Example 1 for mixtures of various sterols and sphingomyelin (1:1) were studied based on the ELISA technique (solid phase technique) described above.

The sterols used herein were cholesterol (cholesterol), ergosterol (ergosterol), lanosterol (lanosterol), coprostane (coprostane), cholesteryl acetate (chol acetate), 5α-cholestane (5a-cholestane), 5α-colestan-3-one (5a-cholestan-3-one), lathosterol (lathosterol, same as 5a-chol-7-en-3b-ol described above), dihydrocholesterol (dihydrocholesterol, same as 5a-chol-3b-ol described above), epicoprostanol (epicoprostanol, same as 5b-chol-3a-ol described above), 6-ketocholestanol (6-ketocholestanol), 7-dehydrocholesterol (7-dehydrocholesterol), 5α-cholest-8(14)-en-3β3-ol-15-one (5a-cholest-8(14)-en-3b-ol-15-one), campesterol (campesterol), stigmasterol (stigmasterol), β-sitosterol (b-sitosterol), desmosterol (desmosterol), epicholesterol (epicholesterol), coprostanol (coprostanol), and coprostenol (coprostenol).

FIG. 13 is a graph showing the binding activity of GF-Nni for the mixtures of various sterols and sphingomyelin. The vertical axis of the graph indicates a relative value wherein the value of cholesterol is set as 100. As shown in FIG. 13, it was found that the 3β-hydroxyl group of the sterol was important in the binding of GF-Nni.

### Example 15

A binding site of GF-Nni on a cell surface was observed by immunoelectron-microscopic analysis. Specifically, the protein GF-Nni expressed and purified by the method described in Example 1 was bound on a fixed HeLa cell surface. Thereafter, the protein was treated with an anti-GF-Nni antibody, then was added a gold colloid-labeled anti-rabbit antibody, and the protein was then observed with an electron microscope.

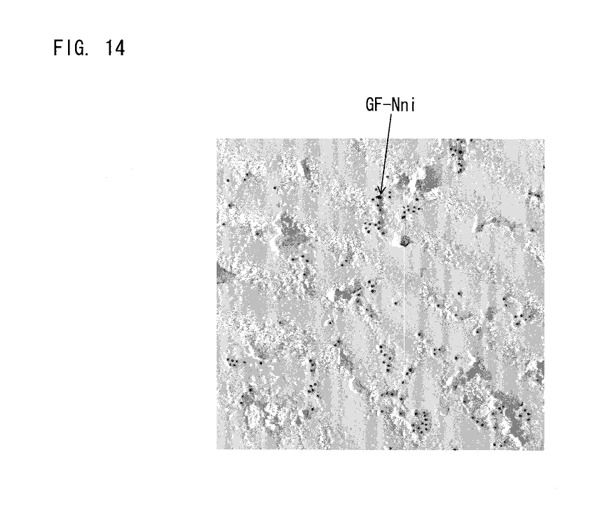

FIG. 14 is a view of a microscopic image showing a binding site of GF-Nni on a surface of a HeLa cell. As shown in FIG. 14, GF-Nni formed a cluster on the cell membrane.

### Example 16

Distribution of GF-Nni, lysenin, H-ras, and K-ras within a cell membrane was observed, by super high resolution fluorescence microscopy. Specifically, GF-Nni, lysenin, H-ras, and K-ras, each of which were labeled with Dronpa and Alexa 647, were prepared, to label the surface of the cell. Thereafter, the labeled cell was observed with use of a PALM (photoactivation localization microscope).

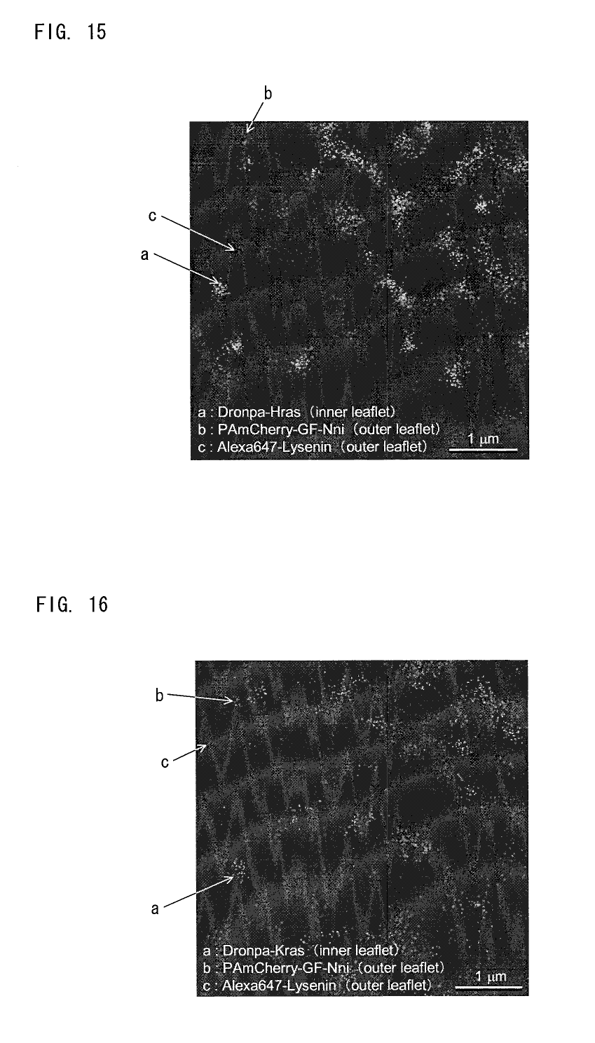

FIG. 15 is a view of a microscopic image showing a distribution of GF-Nni, lysenin, and H-ras within a cell membrane. Moreover, FIG. 16 is a view of a microscopic image showing a distribution of GF-Nni, lysenin, and K-ras within a cell membrane.

It was found in the present Example that the GF-Nni binding domain in a cell membrane is present in a part of a lysenin binding domain. Moreover, GF-Nni distributed in an outer layer of the cell membrane, although matched well with H-ras of the inner layer of the cell membrane, it did not match K-ras. Note that conventionally, H-ras was assumed to be present within a lipid raft. Since the distribution of H-ras matched the distribution of GF-Nni in the present Example, it was shown that H-ras actually was present within a rear side of the sphingomyelin/cholesterol domain.

Use of GF-Nni as such showed that H-ras was actually present within a lipid raft.

### Example 17

Binding activities of GF-Nni for intracellular organelle in a HeLa cell were studied. Specifically, after a cell was fixed, the cell was frozen and thawed to make its membrane permeable. Thereafter, double labeling with various organelle markers and GF-Nni was performed.

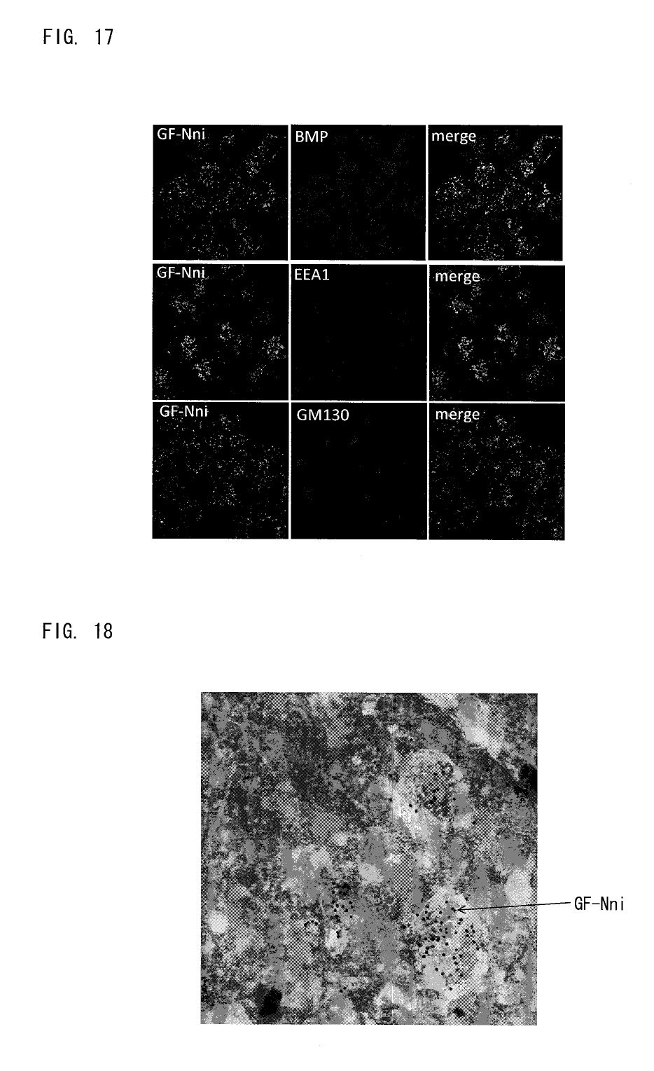

FIG. 17 is a view of a microscopic image illustrating binding activities of GF-Nni for intracellular organelles. As shown in FIG. 17, the localization of GF-Nni only matched BMP, and did not match those of EEA1 and GM130. This result demonstrated that GF-Nni binds to the late endosome within the HeLa cell, thereby showing that the late endosome is a place of accumulating sphingomyelin/cholesterol.

As described above, the use of GF-Nni makes it easy to detect the localization of a lipid raft.

### Example 18

A binding site of GF-Nni within a cell was analyzed by immunoelectron microscopic analysis. Specifically, a section of a Hela cell was prepared and GF-Nni was bound thereto, and thereafter the section was treated with an anti-GF-Nni antibody and a gold colloid-labeled anti-rabbit antibody.

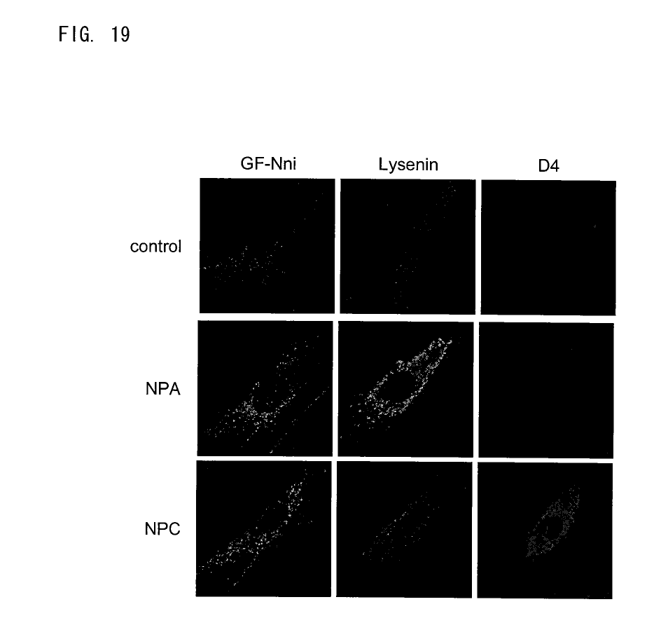

FIG. 18 is a view of a microscopic image showing a binding site of GF-Nni within a cell. This result demonstrated that GF-Nni binds to a multilamellar structure characteristic of a late endosome.

### Example 19

Niemann-Pick disease is a congenital lipid metabolism disorder, and is known to have type A, type C, etc. The type A is caused by a defect of acid sphingomyelinase and the type C is caused by a defect of a protein called Niemann-Pick C protein, which protein is involved in intracellular transport of cholesterol. Cells of the type A cause accumulation of sphingomyelin in the cells, and those of the type C cause accumulation of cholesterol in the cells. Although it was suggested that the function of the lipid raft were abnormal in both types, it was unknown as to the distribution of the lipid raft itself within the cell and on the surface of the cell.

Accordingly, in the present Example, the distribution of lipids inside and on the surface of cells affected by the Niemann-Pick disease was studied with use of lipid binding proteins GF-Nni, lysenin, and D4.

First, to (i) a normal cell (control), (ii) a Niemann-Pick disease type A cell (NPA), and (iii) a Niemann-Pick disease type C cell (NPC), a hole was opened and their membranes were made permeable, by freezing and thawing the cells. Subsequently, GF-Nni, lysenin, and D4 were added thereto, to visualize localizations of GF-Nni, lysenin, and D4 within the cells with use of antibodies against respective proteins.

FIG. 19 is a view of a microscopic image showing a lipid distribution of the cells of Niemann-Pick disease, within the cell. In cells of both the type A and type C of the Niemann-Pick disease, a significant amount of GF-Nni was accumulated within the cell as compared to the control cell. Therefore, the use of GF-Nni showed that the lipid raft was accumulated within the cells of the Niemann-Pick disease.

Next, these cells were fixed, and each of GF-Nni, lysenin, and D4 were bound to surfaces of each of the cells. Thereafter, GF-Nni was visualized by use of an anti-GF-Nni antibody and an Alexa 544-labeled anti-rabbit antibody. Moreover, lysenin and D4 were also visualized similarly by use of a fluorescent antibody.

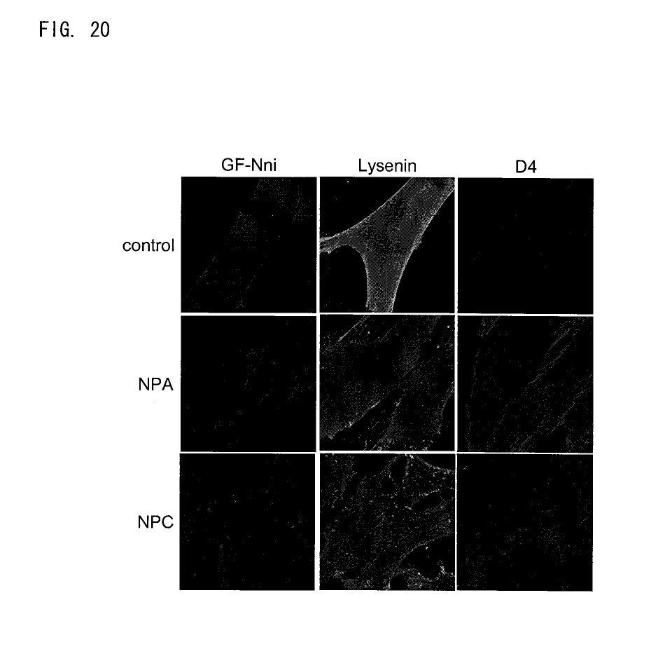

FIG. 20 is a view of a microscopic image showing a lipid distribution on a cell surface of a Niemann-Pick disease cell. It was found that in the type C of the Niemann-Pick disease, the lipid raft (sphingomyelin cholesterol complex) stained by GF-Nni was present on the cell surface as a lump.

As described above, it is possible to perform detailed analysis of diseases such as hereditary diseases associated with a lipid raft, by use of GF-Nni.

### Example 20

It is considered that the lipid raft plays an important role in viral infections including influenza viruses. Accordingly, studies were performed on an effect of GF-Nni on influenza viral infections.

First, effects were observed in cases in which GF-Nni was added to a cultured cell (MDCK) 1 hour before infection with an influenza virus and 4 hours after infection with the influenza virus. More specifically, after treating the cultured cell (MDCK) with GF-Nni for a set period of time, the cultured cell was infected with an influenza virus, and a titer of the virus was measured after a set period of time.

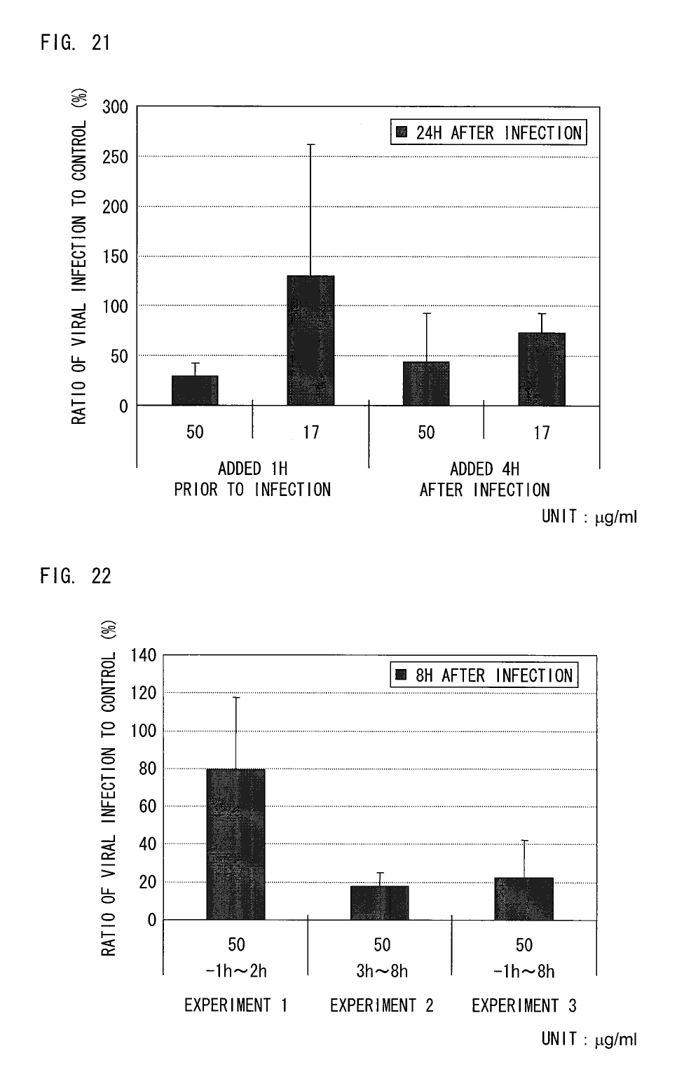

FIG. 21 is a graph showing the effects of GF-Nni on the cultured cell (MDCK) infected with the influenza virus. Note that FIG. 21 shows the result obtained 24 hours after the cell was infected. Moreover, the vertical axis of the graph shows a ratio (%) of viral infection relative to that of the control (MDCK to which no GF-Nni is added). As shown in FIG. 21, the result showed that the viral infection was inhibited more than the control, in both cases of the case in which GF-Nni was added 1 hour prior to the infection, and the case in which GF-Nni was added 4 hours after the infection.

Next, by the method described above, the effects were studied for cases in which periods that the cells and GF-Nni were made in contact with each other were as described below: (i) from 1 hour before the cell was infected with the influenza virus to 2 h.p.i. (experiment 1); (ii) from 3 h.p.i. to 8 h.p.i. (experiment 2); and (iii) from 1 hour before the cell was infected to 8 h.p.i. (experiment 3).

FIG. 22 is a graph showing the effects of GF-Nni on a cultured cell (MDCK) infected with the influenza virus. Note that, FIG. 22 shows a result obtained 8 hours after the infection. Moreover, the vertical axis indicates a ratio (%) of viral infection relative to that of the control (MDCK to which no GF-Nni is added).

As shown in FIG. 22, the inhibition effect on the viral infection was low in experiment 1, where GF-Nni was made into contact with the cell only in the early stage of infection with the influenza virus, i.e., at a time of adsorption/entry to the cell. On the other hand, a high inhibition effect was observed in experiment 2. Therefore, it is assumed that that the point of action of GF-Nni is during a mid to late period of the infection. Moreover, it is suggested that GF-Nni inhibits not the adsorption to the cell of the virus but the release of the virus from a cell.

The above results suggest that GF-Nni can be suitably used for the purpose of inhibiting a viral infection of a cell, for example, as a virus infection inhibitor.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention and the scope of the patent claims set forth below.

Note that the present application is based on the Japanese Patent Application (Japanese Patent Application No. 2010-112681) filed on May 14, 2010, the entire contents of which are hereby incorporated by reference.

### INDUSTRIAL APPLICABILITY

According to the present invention, a lipid raft can be detected specifically. Hence, the present invention can be suitably used in research reagents and kits related to lipid rafts, and further can be suitably used for medicament, virus infection inhibitors for diseases associated with lipid rafts, and the like.

1. A protein being the following (A), (B), or (C):
(A) a protein represented by the amino acid sequence of SEQ ID NO: 1;
(B) a protein represented by an amino acid sequence in which one or a plurality of amino acid is substituted, deleted, inserted or added in the amino acid sequence of SEQ ID NO: 1, the protein having binding activity specific for a mixture of a sphingolipid and cholesterol; or
(C) a protein represented by an amino acid sequence being at least 70% identical to the amino acid sequence of SEQ ID NO: 1, the protein having binding activity specific for a mixture of a sphingolipid and cholesterol.

2. The protein according to claim 1, wherein a labeled polypeptide is added thereto.

3. A polynucleotide coding for a protein recited in claim 1.

4. A polynucleotide being the following (A), (B), (C), or (D):
(A) a polynucleotide represented by the base sequence of SEQ ID NO: 2;
(B) a polynucleotide represented by a base sequence in which one or a plurality of base is substituted, deleted, inserted or added in the base sequence of SEQ ID NO: 2, the polynucleotide coding for a protein having binding activity specific for a mixture of a sphingolipid and cholesterol;
(C) a polynucleotide that hybridizes, under a stringent condition, to a polynucleotide consisting of a base sequence complementary to the base sequence of SEQ ID NO: 2, the polynucleotide coding for a protein having binding activity specific for a mixture of a sphingolipid and cholesterol; or
(D) a polynucleotide represented by a base sequence being at least 70% identical to the base sequence of SEQ ID NO: 2, the polynucleotide coding for a protein having binding activity specific for a mixture of a sphingolipid and cholesterol.

5. A vector comprising a polynucleotide recited in claim 3.

6. A transformant into which a polynucleotide recited in claim 3 is introduced.

7. A transformant into which a vector recited in claim 5 is introduced.

8. An antibody that binds to a protein recited in claim 1.

9. A method of detecting a mixture of a sphingolipid and cholesterol, comprising the step of detecting a mixture of a sphingolipid and cholesterol by use of a protein recited in claim 1.

10. A kit for detecting a lipid raft comprising a protein recited in claim 1.

11. The kit according to claim 10 further comprising at least one of (i) a substance having binding ability specific for sphingomyelin and (ii) a substance having binding activity specific for cholesterol.

12. A protein being a homologue of a protein represented by the amino acid sequence of SEQ ID NO: 1.

13. A virus infection inhibitor comprising a protein recited in claim 1.

14. A kit for detecting a lipid raft comprising a polynucleotide recited in claim 3.

15. The kit according to claim 14 further comprising at least one of (i) a substance having binding ability specific for sphingomyelin and (ii) a substance having binding activity specific for cholesterol.

16. A kit for detecting a lipid raft comprising a vector recited in claim 5.

17. A kit for detecting a lipid raft comprising a transformant recited in claim 6.

18. The kit according to claim 17 further comprising at least one of (i) a substance having binding ability specific for sphingomyelin and (ii) a substance having binding activity specific for cholesterol.

19. A kit for detecting a lipid raft comprising an antibody recited in claim 8.

20. The kit according to claim 19 further comprising at least one of (i) a substance having binding ability specific for sphingomyelin and (ii) a substance having binding activity specific for cholesterol.
